(19) United States
(12) Patent Application Publication
(10) Pub. No.: US 2022/0322801 A1
(43) Pub. Date: Oct. 13, 2022

(54) STORAGE BOX FOR STORING BOTH BLUETOOTH EARPHONE AND ELECTRONIC CIGARETTE

(71) Applicant: LUXSHARE PRECISION INDUSTRY CO., LTD., Shenzhen (CN)

(72) Inventors: Yun FENG, Shenzhen (CN); Huabing LI, Shenzhen (CN); Yu HUANG, Shenzhen (CN)

(73) Assignee: LUXSHARE PRECISION INDUSTRY CO., LTD., Shenzhen (CN)

(21) Appl. No.: 17/526,612

(22) Filed: Nov. 15, 2021

(30) Foreign Application Priority Data
Apr. 12, 2021 (CN) 202120735758.5

(51) Int. Cl. A45C 11/24; A45C 13/02; A61L 2/10; A24F 40/95; H04R 1/10

(52) U.S. Cl. CPC A45C 11/24; A45C 13/02; A61L 2/10; A24F 40/95; H04R 1/1025; A61L 2202/122; A61L 2202/16

(57) ABSTRACT

Provided are a storage box for storing both a bluetooth earphone and an electronic cigarette. The storage box for storing both a bluetooth earphone and the electronic cigarette includes a box body and a box cover covered on a top surface of the box body. The top surface of the box body is provided with an electronic cigarette accommodation groove, a left earphone accommodation groove and a right earphone accommodation groove. The electronic cigarette, the left earphone and the right earphone all are capable of being inserted into the electronic cigarette accommodation groove, the left earphone accommodation groove and the right earphone accommodation groove along the direction perpendicular to the top surface of the box body, respectively.

### CROSS-REFERENCE TO RELATED APPLICATION

This application claims priority to Chinese Patent Application No. 202120735758.5 filed with the CNIPA on Apr. 12, 2021, the disclosure of which is incorporated herein by reference in its entirety.

### TECHNICAL FIELD

The present disclosure relates to the field of storage box and, in particular, to a storage box for storing both a bluetooth earphone and an electronic cigarette.

### BACKGROUND

The true wireless stereo (TWS) technology is developed based on a bluetooth chip technology. The operating principle of the TWS technology is that a mobile phone is connected to a main earphone, and then the main earphone is quickly connected to an auxiliary earphone in a wireless way, so that the real bluetooth left and right channels are wirelessly separated and used. TWS earphones are equipped with a portable box having both charging and storage functions. Some portable boxes also integrate a mobile power supply inside. When the TWS earphones are out of power, just put the earphones into the box, so that the earphones are automatically disconnected and start to be charged.

An electronic cigarette is an electronic product imitating a cigarette, with the same appearance, smoke, taste and feel as the cigarette. In order to facilitate users to carry a bluetooth earphone and an electronic cigarette at the same time, a box for storing both the bluetooth earphone and the electronic cigarette appears in the market. However, the existing box horizontally accommodates the bluetooth earphone and the electronic cigarette, and the minority of the bluetooth earphone and the minority of the electronic cigarette are accommodated in the accommodation groove, so that the bluetooth earphone and the electronic cigarette are placed unstably, and if the box is slightly inclined, the bluetooth earphone and the electronic cigarette are easy to fall out of the box, resulting in inconvenience in use.

Therefore, there is an urgent need for a storage box for storing both a bluetooth earphone and an electronic cigarette to solve the above technique problems.

### SUMMARY

The present disclosure provides a storage box for storing both a bluetooth earphone and an electronic cigarette, which not only improves the stability of accommodating the bluetooth earphone and the electronic cigarette, but also is convenient to take and place, so that the use is simpler.

To achieve this purpose, the present disclosure adopts technical solutions described below.

A storage box for storing both a bluetooth earphone and an electronic cigarette includes a box body and a box cover covered on a top surface of the box body, where the top surface of the box body is provided with an electronic cigarette accommodation groove, a left earphone accommodation groove and a right earphone accommodation groove, the electronic cigarette is capable of being inserted into the electronic cigarette accommodation groove along a direction perpendicular to the top surface of the box body, a left earphone is capable of being inserted into the left earphone accommodation groove along the direction perpendicular to the top surface of the box body, and a right earphone is capable of being inserted into the right earphone accommodation groove along the direction perpendicular to the top surface of the box body. The electronic cigarette accommodation groove, the left earphone accommodation groove and the right earphone accommodation groove all extend along the direction perpendicular to the top surface of the box body.

In an embodiment, a disinfection device is provided in the electronic cigarette accommodation groove.

In an embodiment, the disinfection device is disposed at a bottom end of the electronic cigarette accommodation groove.

In an embodiment, the disinfection device includes an extreme ultraviolet light emitter.

In an embodiment, a battery component is provided in the box body, the left earphone accommodation groove and the right earphone accommodation groove are located at a first side of the battery component, the disinfection device is located at a second side of the battery component, and the first side is opposite to the second side.

In an embodiment, bottom ends of the left earphone accommodation groove and the right earphone accommodation groove are provided with earphone charging ports, a sidewall of the electronic cigarette accommodation groove is provided with an electronic cigarette charging port, and the earphone charging ports and the electronic cigarette charging port are all electrically connected to the battery component.

In an embodiment, a bottom surface of the box body is provided with a battery charging port, and the battery charging port is electrically connected to the battery component.

In an embodiment, a sidewall of the box body is provided with a battery level indicator.

In an embodiment, a sidewall of the box body is provided with an electronic cigarette charging state indicator, a left earphone charging state indicator and a right earphone charging state indicator.

In an embodiment, the electronic cigarette accommodation groove is disposed at a middle position of the top surface of the box body, and the left earphone accommodation groove and the right earphone accommodation groove are located at two sides of the electronic cigarette accommodation groove, respectively.

The present disclosure has the following beneficial effects.

The present disclosure provides a storage box for storing both a bluetooth earphone and an electronic cigarette. The electronic cigarette, the left earphone and the right earphone all are capable of being inserted into the electronic cigarette accommodation groove, the left earphone accommodation groove and the right earphone accommodation groove along the direction perpendicular to the top surface of the box body, respectively. The majority of the electronic cigarette, the majority of the left earphone and the majority of the right earphone are located in the groove, so that the electronic cigarette, the left earphone and the right earphone are placed stably and will not fall out when the box body is inclined at a large angle. In addition, the electronic cigarette, the left earphone and the right earphone can be pulled out by pinching an end part located at a notch during taking. Therefore, the present disclosure provides the storage box for storing both a bluetooth earphone and an electronic cigarette, which improves the stability of accommodating the bluetooth earphone and the electronic cigarette and is convenient to take and place, so that the use is simpler

### BRIEF DESCRIPTION OF DRAWINGS

FIG. 1 is a perspective view of a storage box for storing both a bluetooth earphone and an electronic cigarette according to an embodiment of the present disclosure;

FIG. 2 is a top view of a box body according to an embodiment of the present disclosure;

FIG. 3 is a front view of a storage box for storing both a bluetooth earphone and an electronic cigarette according to an embodiment of the present disclosure; and

FIG. 4 is a bottom view of a storage box for storing both a bluetooth earphone and an electronic cigarette according to an embodiment of the present disclosure.

Reference list
100 electronic cigarette
101 cigarette holder
200 left earphone
201 charging contact
300 right earphone
1 box body
2 box cover
3 disinfection device
4 battery component
11 electronic cigarette accommodation groove
12 left earphone accommodation groove
13 right earphone accommodation groove
14 battery charging port
15 battery level indicator
16 electronic cigarette charging state indicator
17 left earphone charging state indicator
18 right earphone charging state indicator
19 earphone pairing button

### DETAILED DESCRIPTION

Embodiments in accordance with the present disclosure will now be described in detail below. Examples of the embodiments are illustrated in the drawings, where the same or similar reference numerals indicate the same or similar elements or components having the same or similar functions. The embodiments described below with reference to the drawings are exemplary, intended to explain the present disclosure, and not to be construed as limiting the present disclosure.

In the description of the present disclosure, unless otherwise expressly specified and limited, the term “connected to each other”, “connected” or “installed” is to be construed in a broad sense, for example, as installed connected or detachably connected; mechanically connected or electrically connected; directly connected to each other or indirectly connected to each other via an intermediary; or internally connected between two elements or interactional relations between two elements. For those of ordinary skill in the art, specific meanings of the above terms in the present disclosure can be understood according to specific conditions.

In the description of the present disclosure, unless otherwise expressly specified and limited, when a first feature is described as “on” or “below” a second feature, the first feature and the second feature may be in direct contact, or be in contact via another feature between the two features instead of being in direct contact. Moreover, when the first feature is described as “on”, “above” or “over” the second feature, the first feature is right on, above or over the second feature or the first feature is obliquely on, above or over the second feature, or the first feature is simply at a higher level than the second feature. When the first feature is described as “under”, “below” or “underneath” the second feature, the first feature is right under, below or underneath the second feature or the first feature is obliquely under, below or underneath the second feature, or the first feature is simply at a lower level than the second feature.

Solutions of the present disclosure are further described below through embodiments in conjunction with the drawings.

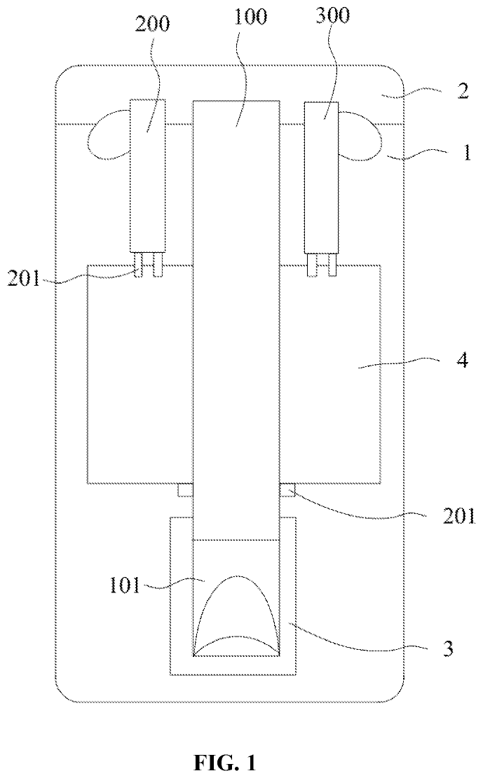

As shown in FIGS. 1 to 4, a storage box for storing both a bluetooth earphone and an electronic cigarette is provided in an embodiment and includes a box body 1 and a box cover 2 covered on a top surface of the box body 1. The top surface of the box body 1 is provided with an electronic cigarette accommodation groove 11, a left earphone accommodation groove 12 and a right earphone accommodation groove 13, the electronic cigarette 100 can be inserted into the electronic cigarette accommodation groove 11 along a direction perpendicular to the top surface of the box body 1, a left earphone 200 can be inserted into the left earphone accommodation groove 12 along the direction perpendicular to the top surface of the box body 1, and a right earphone 300 can be inserted into the right earphone accommodation groove 13 along the direction perpendicular to the top surface of the box body 1.

The embodiment provides the storage box for storing both the bluetooth earphone and the electronic cigarette. The electronic cigarette 100 can be inserted into the electronic cigarette accommodation groove 11 along the direction perpendicular to the top surface of the box body 1, the left earphone 200 can be inserted into the left earphone accommodation groove 12 along the direction perpendicular to the top surface of the box body 1 and the right earphone 300 can be inserted into the right earphone accommodation groove 13 along the direction perpendicular to the top surface of the box body 1. The most part of the electronic cigarette 100, the most part of the left earphone 200 and the most part of the right earphone 300 are located in the groove, so that the electronic cigarette 100, the left earphone 200 and the right earphone 300 are placed stably and will not fall out when the box body 1 is inclined at a large angle. In addition, the electronic cigarette 100, the left earphone 200 and the right earphone 300 can be pulled out by pinching an end part located at a notch during taking. Therefore, the embodiment provides the storage box for storing both a bluetooth earphone and an electronic cigarette, which improves the stability of accommodating the bluetooth earphones and the electronic cigarette and is convenient to take and place, so that the use is simpler.

Specifically, a disinfection device 3 is provided in the electronic cigarette accommodation groove 11. The disinfection device 3 can disinfect the electronic cigarette 100 accommodated in the electronic cigarette accommodation groove 11, especially a cigarette holder 101, thereby ensuring hygiene and safety, and eliminating the consumer's concern about cleanliness when reusing.

More specifically, the disinfection device 3 is provided at the bottom end of the electronic cigarette accommodation groove 11. When the electronic cigarette 100 is placed, the cigarette holder 101 of the electronic cigarette 100 is inserted downward into the bottom end of the electronic cigarette accommodation groove 11, and the disinfection device 3 merely disinfects the cigarette holder 101, which can not only perform the function of disinfection, but also save resources. Furthermore, the cigarette holder 101 facing downward can prevent the hand from touching the cigarette holder 101 and attaching germs again when the electronic cigarette 100 is pulled out again.

Specifically, the disinfection device 3 includes an extreme ultraviolet light emitter. The cigarette holder 101 is irradiated by the extreme ultraviolet light emitted by the extreme ultraviolet light emitter, and the germs on the cigarette holder 101 are killed. Apparently, the disinfection device 3 is not limited to the extreme ultraviolet light emitter, but may also be another type of sterilizable light emitter, and may also be a disinfection device spraying a disinfection spray.

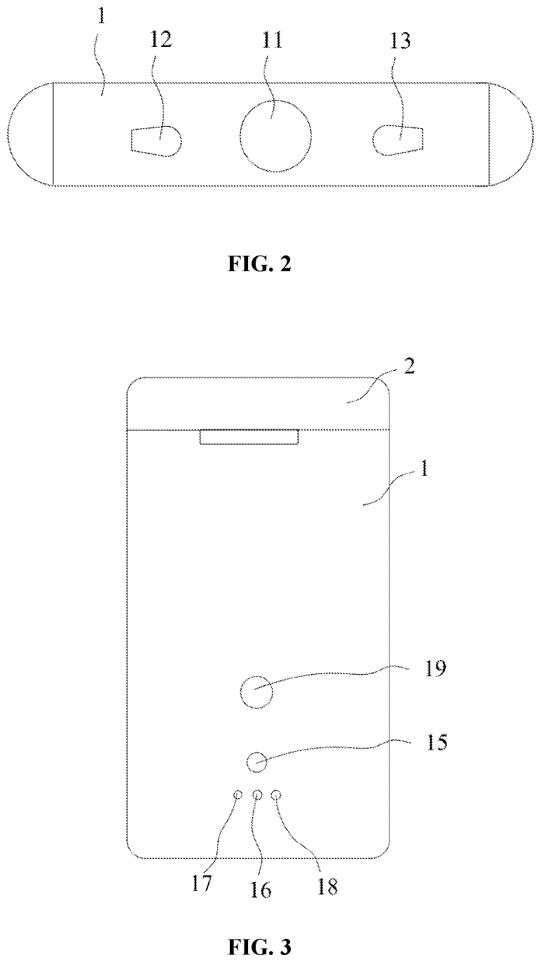

As shown in FIGS. 1 and 2, a battery component 4 is provided in the box body 1, the left earphone accommodation groove 12 and the right earphone accommodation groove 13 are located at a first side of the battery component 4, the disinfection device 3 is located at a second side of the battery component 4, and the first side is opposite to the second side. At the orientation shown in FIG. 1, the left earphone accommodation groove 12 and the right earphone accommodation groove 13 are located above the battery component 4, the bottom end of the electronic cigarette accommodation groove 11 extends below the battery component 4, that is, the disinfection device 3 is located below the battery component 4. This arrangement makes the overall structure of the components in the box body 1 compact, saves space, facilitates the electrical connection between each groove and the battery component 4, and facilitates the charging of the electronic cigarette 100, the left earphone 200 and the right earphone 300.

Specifically, bottom ends of the left earphone accommodation groove 12 and the right earphone accommodation groove 13 are provided with earphone charging ports, a sidewall of the electronic cigarette accommodation groove 11 is provided with an electronic cigarette charging port, and the earphone charging ports and the electronic cigarette charging port both are electrically connected to the battery component 4. The left earphone accommodation groove 12 and the right earphone accommodation groove 13 are located above the battery component 4. The earphone charging ports are disposed at the bottom ends of the left earphone accommodation groove 12 and the right earphone accommodation groove 13, which is convenient for the layout of connection lines. When the left earphone 200 is inserted into place in the left earphone accommodation groove 12, a charging contact 201 at a rear end of the left earphone 200 is inserted into an earphone charging port to achieve electrical connection between the left earphone 200 and the battery component 4 so as to charge the left earphone 200. The charging of the right earphone 300 is the same as the charging of the left earphone 200, which will not be described here. Similarly, when the electronic cigarette 100 is inserted into the electronic cigarette accommodation groove 11, the charging contact 201 on the electronic cigarette 100 is inserted into the electronic cigarette charging port to charge the electronic cigarette 100.

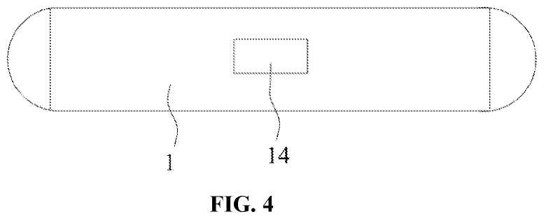

The battery component 4 includes a battery and a control circuit module electrically connected to the battery, and the control circuit module controls charging and discharging of the battery. In order to charge the battery, as shown in FIG. 4, a bottom surface of the box body 1 is provided with a battery charging port 14, and the battery charging port 14 is electrically connected to the battery component 4. The battery is connected to an external power supply through the battery charging port 14 by a matched charger, and the battery is charged under the action of the control circuit module. The control of the battery by the above-mentioned control circuit module is a conventional setting in the present field, and is not described herein again.

As shown in FIG. 3, a sidewall of the box body 1 is provided with a battery level indicator 15. The battery level indicator 15 is used for displaying the amount of power in the battery component 4 to remind the user to charge in time. In the embodiment, the control circuit module displays whether the battery power is sufficient by controlling the light on and off, that is, when the battery level indicator 15 is off, it indicates that the power is sufficient; when the battery level indicator 15 is on, it indicates that the power is insufficient, and the color may be red when the battery level indicator 15 is on.

Specifically, a sidewall of the box body 1 is provided with an electronic cigarette charging state indicator 16, a left earphone charging state indicator 17 and a right earphone charging state indicator 18. In the embodiment, when the electronic cigarette charging state indicator 16 is on, it indicates that the electronic cigarette 100 is in a charging state. After the electronic cigarette charging state indicator 16 is on for a certain period of time, if the electronic cigarette 100 is still in the electronic cigarette accommodation groove 11 and the electronic cigarette charging state indicator 16 is off, it indicates that the electronic cigarette 100 is fully charged. Similarly, the operation states of the left earphone charging state indicator 17 and the right earphone charging state indicator 18 are the same as the operation state of the electronic cigarette charging state indicator 16 described above, and will not be repeated here.

In an embodiment, a sidewall of the box body 1 is provided with an earphone pairing button 19, and the left earphone 200 and the right earphone 300 can be paired by pressing the earphone pairing button 19.

In an embodiment, the electronic cigarette accommodation groove 11 is disposed at a middle position of the top surface of the box body 1, and the left earphone accommodation groove 12 and the right earphone accommodation groove 13 are located at two sides of the electronic cigarette accommodation groove 11, respectively. This arrangement makes the structure more compact. In this embodiment, the left earphone accommodation groove 12 and the right earphone accommodation groove 13 are symmetrically disposed with respect to the electronic cigarette accommodation groove 11, so that the storage box as a whole is of a symmetrical structure and is more convenient to use.

Specifically, on a side surface of the box body 1, the electronic cigarette charging state indicator 16 is located between the left earphone charging state indicator 17 and the right earphone charging state indicator 18, and the left earphone charging state indicator 17 and the right earphone charging state indicator 18 are symmetrically disposed with respect to the electronic cigarette charging state indicator 16. As shown in FIGS. 2 and 3, the left earphone charging state indicator 17 is located on a left side of the electronic cigarette charging state indicator 16. Correspondingly, the left earphone accommodation groove 12 is located on a left side of the electronic cigarette accommodation groove 11, and the right earphone charging state indicator 18 is located on a right side of the electronic cigarette charging state indicator 16. Correspondingly, the right earphone accommodation groove 13 is located on a right side of the electronic cigarette accommodation groove 11.

Specifically, one end of the box cover 2 is rotatably connected to one end of the top surface of the box body 1, and the other end of the box cover 2 is snapped to the other end of the top surface of the box body 1 through a snap. The box body 1 is opened or closed by overturning the box cover 2, which is convenient and quick to take and place the bluetooth earphone and the electronic cigarette.

Apparently, the above embodiments of the present disclosure are merely examples to illustrate the present disclosure and are not intended to limit embodiments of the present disclosure. For those of ordinary skill in the art, alterations or modifications in other different forms can be made based on the above description. Implementations of the present disclosure cannot be and do not need to be all exhausted herein. Any modifications, equivalent substitutions and improvements within the spirit and principle of the present disclosure fall within the scope of the claims of the present disclosure.

What is claimed is:

1. A storage box for storing both a bluetooth earphone and an electronic cigarette, comprising a box body and a box cover covered on a top surface of the box body, wherein the top surface of the box body is provided with an electronic cigarette accommodation groove, a left earphone accommodation groove and a right earphone accommodation groove, an electronic cigarette is capable of being inserted into the electronic cigarette accommodation groove along a direction perpendicular to the top surface of the box body, a left earphone is capable of being inserted into the left earphone accommodation groove along the direction perpendicular to the top surface of the box body, and a right earphone is capable of being inserted into the right earphone accommodation groove along the direction perpendicular to the top surface of the box body.

2. The storage box for storing both a bluetooth earphone and an electronic cigarette of claim 1, wherein a disinfection device is provided in the electronic cigarette accommodation groove.

3. The storage box for storing both a bluetooth earphone and an electronic cigarette of claim 2, wherein the disinfection device is disposed at a bottom end of the electronic cigarette accommodation groove.

4. The storage box for storing both a bluetooth earphone and an electronic cigarette of claim 2, wherein the disinfection device comprises an extreme ultraviolet light emitter.

5. The storage box for storing both a bluetooth earphone and an electronic cigarette of claim 2, wherein a battery component is provided in the box body, the left earphone accommodation groove and the right earphone accommodation groove are located at a first side of the battery component, the disinfection device is located at a second side of the battery component, and the first side is opposite to the second side.

6. The storage box for storing both a bluetooth earphone and an electronic cigarette of claim 5, wherein bottom ends of the left earphone accommodation groove and the right earphone accommodation groove are provided with earphone charging ports, a sidewall of the electronic cigarette accommodation groove is provided with an electronic cigarette charging port, and the earphone charging ports and the electronic cigarette charging port are all electrically connected to the battery component.

7. The storage box for storing both a bluetooth earphone and an electronic cigarette of claim 5, wherein a bottom surface of the box body is provided with a battery charging port, and the battery charging port is electrically connected to the battery component.

8. The storage box for storing both a bluetooth earphone and an electronic cigarette of claim 1, wherein a sidewall of the box body is provided with a battery level indicator.

9. The storage box for storing both a bluetooth earphone and an electronic cigarette of claim 1, wherein a sidewall of the box body is provided with an electronic cigarette charging state indicator, a left earphone charging state indicator and a right earphone charging state indicator.

10. The storage box for storing both a bluetooth earphone and an electronic cigarette of claim 1, wherein the electronic cigarette accommodation groove is disposed at a middle position of the top surface of the box body, and the left earphone accommodation groove and the right earphone accommodation groove are located at two sides of the electronic cigarette accommodation groove, respectively.

11. The storage box for storing both a bluetooth earphone and an electronic cigarette of claim 2, wherein a sidewall of the box body is provided with a battery level indicator.

12. The storage box for storing both a bluetooth earphone and an electronic cigarette of claim 3, wherein a sidewall of the box body is provided with a battery level indicator.

13. The storage box for storing both a bluetooth earphone and an electronic cigarette of claim 4, wherein a sidewall of the box body is provided with a battery level indicator.

14. The storage box for storing both a bluetooth earphone and an electronic cigarette of claim 5, wherein a sidewall of the box body is provided with a battery level indicator.

15. The storage box for storing both a bluetooth earphone and an electronic cigarette of claim 6, wherein a sidewall of the box body is provided with a battery level indicator.

16. The storage box for storing both a bluetooth earphone and an electronic cigarette of claim 7, wherein a sidewall of the box body is provided with a battery level indicator.

17. The storage box for storing both a bluetooth earphone and an electronic cigarette of claim 2, wherein a sidewall of the box body is provided with an electronic cigarette charging state indicator, a left earphone charging state indicator and a right earphone charging state indicator.

18. The storage box for storing both a bluetooth earphone and an electronic cigarette of claim 3, wherein a sidewall of the box body is provided with an electronic cigarette charging state indicator, a left earphone charging state indicator and a right earphone charging state indicator.

19. The storage box for storing both a bluetooth earphone and an electronic cigarette of claim 4, wherein a sidewall of the box body is provided with an electronic cigarette charging state indicator, a left earphone charging state indicator and a right earphone charging state indicator.

20. The storage box for storing both a bluetooth earphone and an electronic cigarette of claim 5, wherein a sidewall of the box body is provided with an electronic cigarette charging state indicator, a left earphone charging state indicator and a right earphone charging state indicator.
